(19) United States
(12) Patent Application Publication
(10) Pub. No.: US 2007/0208331 A1
(43) Pub. Date: Sep. 6, 2007

(54) Systems and methods of treatment within a gastrointestinal lumen

(76) Inventors: Michael S.H. Chu, Brookline, MA (US); Paul DiCarlo, Middleboro, MA (US); Issac Ostrovsky, Wellesley, MA (US); Robert F. Rioux, Ashland, MA (US); Ashley Seehusen, Newton, MA (US); William J. Shaw, Cambridge, MA (US); Vincent A. Turturro, Bolton, MA (US)

Correspondence Address: COOLEY GODWARD KRONISH LLP;ATTN: PATENT GROUP, Suite 500, 1200 - 19th Street, NW, WASHINGTON, DC, 20036-2402, US

(21) Appl. No.: 11/365,919

(22) Filed: Mar. 2, 2006

(51) Int. Cl. A61B 18/04

(52) U.S. Cl. 606/32

(57) ABSTRACT

A method includes identifying an area of interest within a gastrointestinal lumen based on a virtual imaging modality. Energy is applied using an external energy source directed at the area of interest sufficient to disrupt at least a portion of an undesired tissue. In another embodiment, the method includes introducing a contrasting agent into a gastrointestinal lumen. The gastrointestinal lumen is imaged with an imaging device. An area of interest within the gastrointestinal lumen is identified, where the area of interest is indicated by the contrasting agent. Energy is applied using an external energy source directed at the area of interest to disrupt at least a portion of a tissue located at the area of interest. The disrupted tissue is removed from the gastrointestinal lumen.

### BACKGROUND

The present invention relates generally to medical procedures for use in conjunction with an external non-invasive imaging procedure, and more particularly to systems and methods of treatment within a gastrointestinal lumen performed in conjunction with an external non-invasive imaging procedure.

Colorectal cancer is one of the leading causes of deaths from malignancy in the United States, with only lung cancer causing more deaths annually. Colon cancer can be prevented because it usually begins as a benign polyp that grows slowly for several years before becoming cancerous. If polyps are detected and removed, the risk of developing colon cancer is significantly reduced.

Unfortunately, widespread colorectal screening and preventive efforts are hampered by several practical impediments, including limited resources, methodologic inadequacies, and poor patient acceptance leading to poor compliance. Moreover, some tests, such as the fecal occult blood test (FOBT) fail to detect the majority of cancers and pre-cancerous polyps. Additionally, since a sigmoidoscopy only examines a portion of the colon, it also misses many polyps that occur in the remainder of the colon. The accuracy of other tests, such as the barium enema, vary and are not always reliable.

A technique for detecting colorectal cancer using helical computed tomography (CT) to create computer simulated intraluminal flights through the colon was proposed as a novel approach for detecting colorectal neoplasms by Vining D J, Shifrin R Y, Grishaw E K, Liu K, Gelfand D W, Virtual colonoscopy (Abst), Radiology Scientific Prgm 1994; 193(P):446. This technique was first described by Vining et al. in an earlier abstract by Vining D J, Gelfand D W, Noninvasive colonoscopy using helical CT scanning, 3D reconstruction, and virtual reality (Abst), SGR Scientific Program, 1994. This technique, referred to as “virtual colonoscopy”, requires a cleansed colon insufflated with air, a helical CT scan of approximately 30 seconds, and specialized three-dimensional (3D) imaging software to extract and display the mucosal surface. The resulting endoluminal images generated by the CT scan are displayed to a medical practitioner for diagnostic purposes.

There have been several advances in virtual colonoscopy that have improved the imaging techniques, making it a more viable and effective screening option. One advantage of using a virtual colonoscopy as a screening process is the reduction of the invasiveness of a traditional colonoscopy. Traditional colonoscopies are preformed using a colonoscope that has a relatively large diameter (i.e., sufficient to form a seal with the anus) that includes, among other instruments, a scope, multiple lumens for introducing gas and/or liquid, and a working channel for introducing a snare or similar device into the colon. With such a device, there is a risk of straightening and/or perforating the colon because of its relative inflexibility and size.

Another advantage of the virtual colonoscopy procedure is the elimination of the preparation process associated with a traditional colonoscopy. The typical preparation process involves the use of strong laxatives to purge any fecal waste from the colon. Such a process is extremely uncomfortable and is often cited as one of the least desirable parts of the whole procedure. Complete purging is not necessary with the virtual colonoscopy procedure. Rather, a fecal contrasting agent is used to facilitate digital subtraction of any residual feces from the virtual image.

Even though the virtual colonoscopy is largely non-invasive as a screening process, a need still exists for non-invasive and minimally invasive devices and methods for treating the colon (e.g., removing polyps) in the event the virtual colonoscopy identifies a problem area within the colon.

Traditional devices such as colonoscopes or snares are used to dislodge and remove polyps from the colon that have been identified with a virtual colonoscopy. Colonoscopes have a relatively large diameter, limited flexibility and typically include multiple instruments, such as a passageway to insufflate the colon, a scope and a working tool. Due to the risks associated with such a device, such as straightening and/or perforating the colon, there is a need for other options and methods to disrupt a polyp within the colon using a less invasive instrument. Thus, there is a need for a method of treatment that provides a means for disrupting a polyp or other undesirable tissue, and/or treating an affected area within a gastrointestinal lumen, in conjunction with a virtual imaging modality.

### SUMMARY OF THE INVENTION

A method includes identifying an area of interest within a gastrointestinal lumen based on a virtual imaging modality. Energy is applied using an external energy source directed at the area of interest sufficient to disrupt at least a portion of an undesired tissue. In another embodiment, the method includes introducing a contrasting agent into a gastrointestinal lumen. The gastrointestinal lumen is imaged with an imaging device. An area of interest within the gastrointestinal lumen is identified, where the area of interest is indicated by the contrasting agent. Energy is applied using an external energy source directed at the area of interest to disrupt at least a portion of a tissue located at the area of interest. The disrupted tissue is removed from the gastrointestinal lumen.

### BRIEF DESCRIPTION OF THE DRAWINGS

The present invention is described with reference to the accompanying drawings. In the drawings, like reference numbers indicate identical or functionally similar elements.

FIG. 1 is an illustration of a large intestine.

FIGS. 2A and 2B illustrate different types of polyps in a colon.

FIG. 3 is a schematic illustration of devices used in a medical procedure according to an embodiment of the invention.

FIG. 4 is a plan view of an apparatus according to an embodiment of the invention shown positioned within a large intestine.

FIG. 5 is a flow chart of a method according to an embodiment of the invention.

FIG. 6 is a flow chart of a method according to an embodiment of the invention.

### DETAILED DESCRIPTION

A method includes identifying an area of interest within a gastrointestinal lumen based on a virtual imaging modality. Energy is applied using an external energy source directed at the area of interest sufficient to disrupt at least a portion of an undesired tissue. In another embodiment, the method includes introducing a contrasting agent into a gastrointestinal lumen. The gastrointestinal lumen is imaged with an imaging device. An area of interest within the gastrointestinal lumen is identified, where the area of interest is indicated by the contrasting agent. Energy is applied using an external energy source directed at the area of interest to disrupt at least a portion of a tissue located at the area of interest. The disrupted tissue is removed from the gastrointestinal lumen.

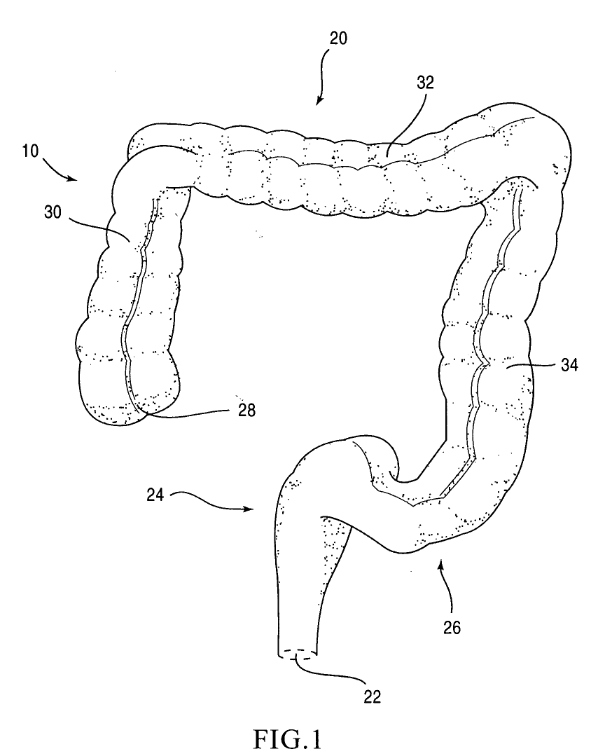

Referring to FIG. 1, an illustration of a large intestine (also called the large bowel) 10 is provided by way of background and reference. The colon 20 is the longest part of the large intestine 10, which is a tube-like organ connected to the small intestine (not illustrated) at one end and the anus 22 at the other. The colon 20 and the rectum 24 form the large intestine 10. The colon 20 is the first 4 to 5 feet of the large intestine 10, and the rectum 24 is the last 4 to 5 inches. The part of the colon 20 that joins to the rectum 24 is called the sigmoid colon 26. The junction of the two parts is often referred to as the rectosigmoid colon or rectosigmoid process. The part of the colon 20 that joins to the small intestine is called the cecum 28. The cecum 28 is adjacent the ascending colon 30, which is connected to the transverse colon 32. The transverse colon 32 is connected to the descending colon 34, which is connected to the sigmoid colon 26. The colon 20 removes/absorbs water and some nutrients and electrolytes from partially digested food. The remaining material, solid waste, called stool or feces, moves through the colon 20 to the rectum 24 and leaves the body through the anus 22.

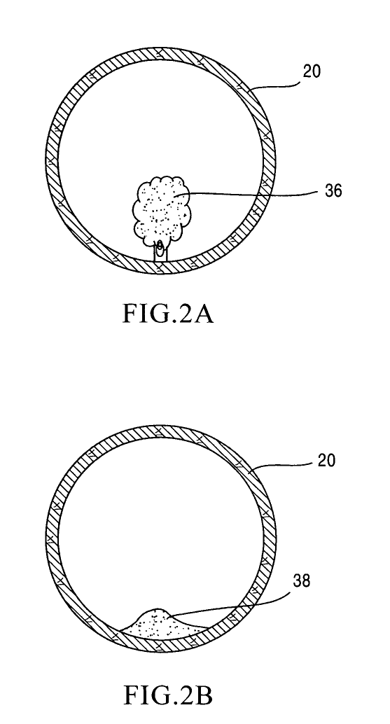

FIGS. 2A-2B illustrate various types of polyps that can form in the colon. A gastrointestinal polyp is a mass of the mucosal surface of the intestine that protrudes into the passageway of the bowel. Polyps can be neoplastic, non-neoplastic, or submucosal. Adenomatous polyps are abnormal growths in the colon and are more likely to develop into or already contain cancer than other types of colon polyps. Adenomatous polyps, however, usually contain tissue that is abnormal but not necessarily cancerous, hence the importance of being able to completely remove a polyp from the colon. The size, type of tissue, and degree of abnormality (mild, moderate, or severe) in a polyp determines the likelihood that it contains cancer.

Some adenomatous polyps are attached to the wall of the colon or rectum by a stalk (a pedunculated polyp 36) as illustrated in FIG. 2A. Some polyps have a broad base with little or no stalk (a sessile polyp 38) as illustrated in FIG. 2B.

The system and methods of the present invention involve the use of known external treatment methods, such as ultrasound and radiation therapy, used in conjunction with a virtual imaging modality, such as a virtual colonoscopy. Although the below description focuses primarily on polyp removal from a colon, the methods and medical procedures described can be used in other gastrointestinal lumens. In addition, the methods and procedures are not limited to the removal of polyps. For example, the described methods can be used to disrupt tumors and treat other forms of undesirable tissue within a gastrointestinal lumen.

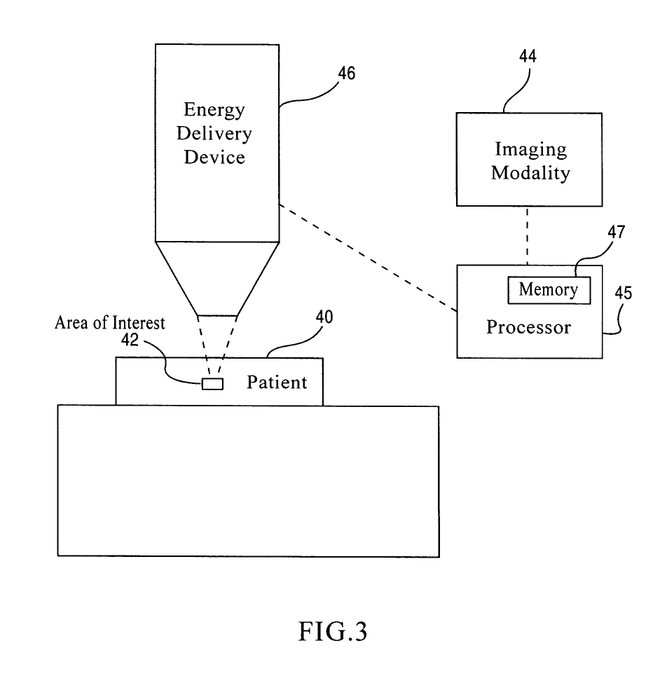

FIG. 3 is a schematic illustration of devices used in a non-invasive method of disrupting and removing an undesirable tissue, such as a polyp or tumor, from a gastrointestinal lumen. An image of a patient's colon or other gastrointestinal lumen can be taken by at least one of a variety of imaging modalities 44, such as a virtual colonoscopy accomplished with a computed tomography (CT) device, or a magnetic resonance imaging (MRI) device. A virtual colonoscopy can be used, for example, to emulate an internal method of imaging, such as using an endoscope. Thus, a virtual colonoscopy is one form of non-invasive imaging method. Other non-invasive imaging methods can alternatively be used.

An area of interest 42 in a patient 40 having undesirable tissue, such as a polyp, present within the colon (or other gastrointestinal lumen) can be identified by the image created by the imaging modality 44. After the area of interest 42 is identified using the imaging modality 44, the area of interest 42 can be treated using an external energy delivery device 46 that is directed at the area of interest. Alternatively, the image can be taken contemporaneously with the treatment of the area of interest. For example, a physician can use an image produced with a virtual colonoscopy to contemporaneously guide the treatment of the area of interest. The energy delivery device 46 can include an energy source used to treat the area of interest 42, such as, for example, acoustic energy (e.g., ultrasound) or electromagnetic energy (e.g., microwave, ionizing radiation). If the area of interest 42 includes undesirable tissue, such as a polyp, the energy can be directed at the polyp, which effectively kills the polyp and disrupts it from its connection to the colon wall. The disrupted polyp can then be removed from the colon using known polyp removal procedures such as the use of a suctioning device coupled to an endoscope, or the use of a snare.

In some embodiments a system is provided with the imaging modality 44 in communication with the energy delivery device 46, through, for example, a processor 45. The processor 45 can be, for example, a commercially available personal computer, or a less complex computing or processing device that is dedicated to performing one or more specific tasks. The processor 45, according to one or more embodiments of the invention, can be a commercially available microprocessor. Alternatively, the processor 45 can be an application-specific integrated circuit (ASIC) or a combination of ASICs, which are designed to achieve one or more specific functions, or enable one or more specific devices or applications. In yet another embodiment, the processor 45 can be an analog or digital circuit, or a combination of multiple circuits.

The processor 45 can include a memory component 47. The memory component 47 can include one or more types of memory. The processor 45 can store data in the memory component 47 or retrieve data previously stored in the memory component 47. The components of the processor 45 can communicate with devices external to the processor 45 by way of an input/output (I/O) component (not shown). According to one or more embodiments of the invention, the I/O component can include a variety of suitable communication interfaces.

The use of ultrasound technology for imaging purposes is well known. Developments in ultrasound technology have provided for the use of ultrasound technology not only for imaging, but for therapeutic and surgical procedures as well. For use in these types of procedures, the ultrasound is provided at very high frequencies to raise the temperature of the directed ultrasonic energy. The high-powered ultrasound can be focused to a particular area to be treated without affecting areas outside the directed focal point. This high intensity focused ultrasound (HIFU) can be used to kill tissue (e.g., polyps, cancer cells) and to stop internal bleeding by cauterizing injured organs or blood vessels. Electromagnetic energy (e.g., radiation therapy) is also used to kill cancer cells and treat tumors by applying the energy external to the body of the patient. Similar to the ultrasound energy treatment, improvements have been made with electromagnetic treatment techniques that allow the energy to be targeted to the area to be treated with minimal effects to surrounding healthy tissue.

In an embodiment where acoustic energy (e.g., ultrasound) is used as the source of energy, the gastrointestinal lumen can be insufflated with an acoustic coupling medium, such as water, prior to applying the external energy to the area of interest 42. The acoustic energy will be transmitted to the area of interest 42 via the acoustic coupling medium.

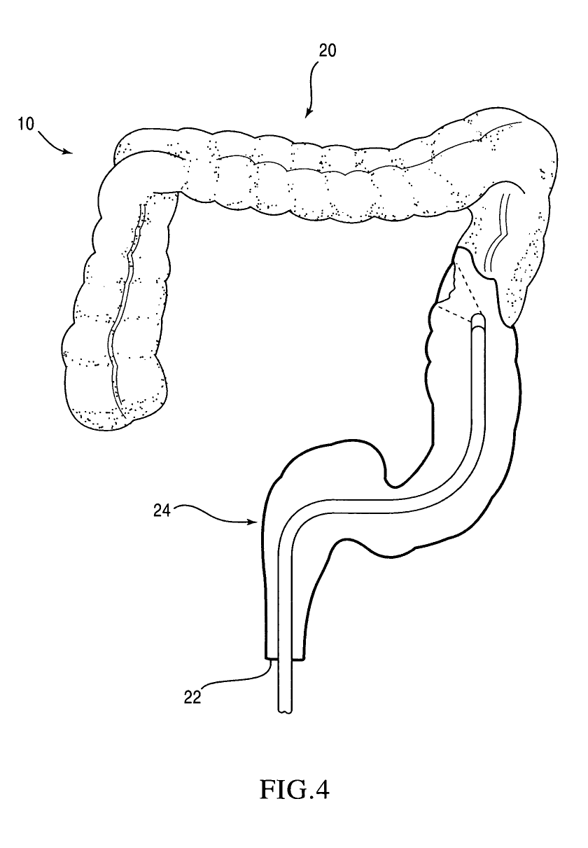

FIG. 4 illustrates a device associated with an alternative method of treating an area of interest within a gastrointestinal lumen. An image of the gastrointestinal lumen can be taken using a virtual imaging modality as described above. An area of interest 76, such as a polyp, can then be identified from the image. An energy probe 78 coupled to a colonoscope 80, or other suitable device, can be positioned proximate to the area of interest 76 to be treated. Electromagnetic energy (e.g., radiation) can then be applied and directed at the area of interest 76 to disrupt the undesired tissue, or otherwise treat the area of interest 76.

In some embodiments, a contrasting agent, such as barium sulphate or Gastrografin® (sodium amidotrizoate), can be introduced into the gastrointestinal lumen prior to imaging the gastrointestinal lumen. The contrasting agent is configured to alter the appearance of the undesirable tissue when imaged, such as a polyp, to help identify the area of interest. The area of interest can then be treated by applying energy to the area of interest either with an external energy source or using an internal energy probe as described above.

The methods and apparatuses disclosed herein can further include the use of protective or targeted approaches to direct the energy to the correct area of tissue. For example, ex vivo or in vivo lenses, masks or protective agents applied to tissue will prevent healthy tissue from being affected.

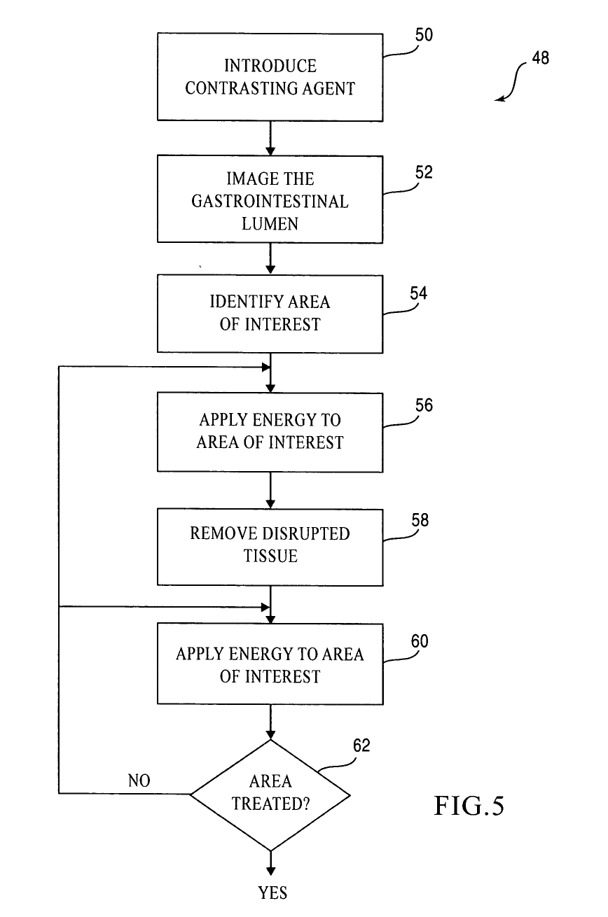

FIG. 5 is a flowchart of a method 48 according to the invention. A contrasting agent can be introduced into a gastrointestinal lumen, such as a colon, at step 50. An image of the gastrointestinal lumen can then be taken using an external imaging modality such as a CT or MRI at step 52. An area of interest is identified from the image at step 54. Energy is applied externally to the patient at step 56, and directed at the area of interest within the patient's body. As described above, the energy can be acoustic energy, electromagnetic energy or other suitable energy. In alternative embodiments, the energy can be administered to the area of interest by placing a probe within the gastrointestinal lumen and positioned proximate to the area of interest to be treated as described above. The applied energy can be used to disrupt or dislodge undesirable tissue connected to the gastrointestinal lumen walls. The disrupted or dislodged tissue can then be removed from the gastrointestinal lumen at step 58 using known removal procedures. After the undesirable tissue has been dislodged or removed, energy can be applied again to the area of interest at step 60, if necessary, to further treat the affected area of interest. This may be desired, for example, to treat an area where a polyp has been removed that may require further treatment to the underlying gastrointestinal lumen walls. If the area of interest requires even further treatment, the process of applying energy can be repeated at step 56 or 60.

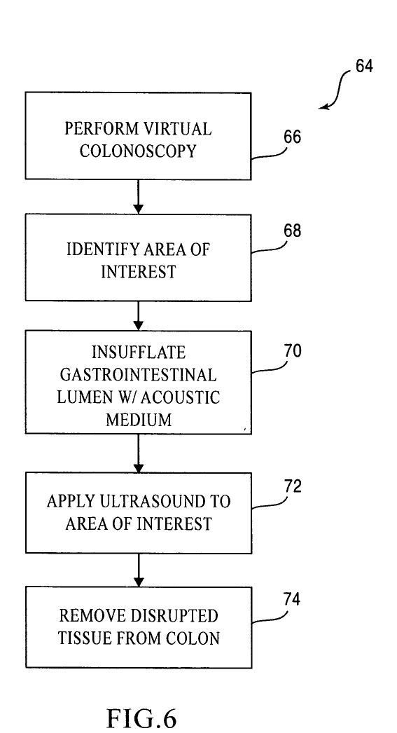

FIG. 6 is a flowchart of another method of treating undesired tissue (e.g., a polyp) within a colon according to an embodiment of the invention. A method 64 includes performing a virtual colonoscopy utilizing an imaging modality such as a CT or MRI device at step 66. An area of interest, such as a polyp is identified from the image at step 68. The gastrointestinal lumen or colon is insufflated with an acoustic coupling medium, such as water, at step 70 using known insufflation devices and techniques. Acoustic energy (e.g., ultrasound) is applied externally to the patient, and is transmitted to the area of interest via the acoustic coupling medium to disrupt or dislodge the undesired tissue at step 72. The undesired tissue can then be removed from the colon using known extraction techniques at step 74.

### CONCLUSION

While various embodiments of the invention have been described above, it should be understood that they have been presented by way of example only, and not limitation. Thus, the breadth and scope of the invention should not be limited by any of the above-described embodiments, but should be defined only in accordance with the following claims and their equivalents.

The previous description of the embodiments is provided to enable any person skilled in the art to make or use the invention. While the invention has been particularly shown and described with reference to embodiments thereof, it will be understood by those skilled in art that various changes in form and details may be made therein without departing from the spirit and scope of the invention.

For example, the procedures and methods described herein may be used to treat various medical conditions within a gastrointestinal lumen. The applied energy can be directed at the area of interest externally to the patient, or using a probe positioned proximate the area of interest. The type of energy delivered is not limited to ultrasound. Other types of energy, such as electromagnetic radiation, with or without a coupling medium can be used.

What is claimed is:

1. A method, comprising:
identifying an area of interest within a gastrointestinal lumen based on an external non-invasive imaging modality; and
applying energy using an external energy source directed at the area of interest sufficient to disrupt at least a portion of an undesired tissue.

2. The method of claim 1, wherein the applying the energy includes applying at least one of acoustic energy and electromagnetic energy.

3. The method of claim 1, wherein the applying the energy includes applying acoustic energy, the method further comprising:
prior to the applying energy, insufflating the gastrointestinal lumen with an acoustic coupling medium.

4. The method of claim 1, further comprising:
removing the disrupted tissue from the gastrointestinal lumen.

5. The method of claim 1, wherein the identifying an area of interest includes imaging the gastrointestinal lumen using a computed tomography scanner.

6. The method of claim 1, wherein the identifying an area of interest includes imaging the gastrointestinal lumen using a magnetic resonance imaging device.

7. The method of claim 1, wherein the external non-invasive imaging modality includes a virtual imaging modality.

8. A method, comprising:
introducing a contrasting agent into a gastrointestinal lumen;
imaging the gastrointestinal lumen with an imaging device;
identifying an area of interest within the gastrointestinal lumen, the area of interest indicated by the contrasting agent;
applying energy using an external energy source directed at the area of interest to disrupt at least a portion of a tissue located at the area of interest; and
removing the disrupted tissue from the gastrointestinal lumen.

9. The method of claim 8, further comprising:
applying energy using an external energy source directed at the area of interest after the disrupted tissue has been removed.

10. The method of claim 8, wherein the identifying the area of interest includes imaging the gastrointestinal lumen with a computed tomography scanner.

11. The method of claim 8, wherein the identifying the area of interest includes imaging the gastrointestinal lumen with a magnetic resonance imaging device.

12. The method of claim 8, wherein the applying the energy includes applying at least one of acoustic energy and electromagnetic energy.

13. The method of claim 8, further comprising:
prior to the applying energy, insufflating the gastrointestinal lumen with an acoustic coupling medium and applying the energy includes using a source of acoustic energy and transmitting the acoustic energy to the area of interest via the acoustic coupling medium.

14. A method, comprising:
creating an image of a gastrointestinal lumen of a patient based on a virtual imaging modality;
identifying an area of interest within the gastrointestinal lumen;
inserting an energy probe into the gastrointestinal lumen proximate to the area of interest; and
applying energy via the energy probe, the energy being directed at the area of interest and being sufficient to disrupt undesired tissue located at the area of interest.

15. The method of claim 14, further comprising:
removing the energy probe; and
removing the disrupted tissue from the gastrointestinal lumen.

16. The method of claim 14, wherein the applying the energy includes applying at least one of acoustic energy and electromagnetic energy.

17. The method of claim 14, wherein the imaging a gastrointestinal lumen includes using a computed tomography scanner.

18. The method of claim 14, wherein the imaging a gastrointestinal lumen includes using a magnetic resonance imaging device.

19. The method of claim 14, further comprising:
introducing a contrasting agent into the gastrointestinal lumen prior to imaging the gastrointestinal lumen.

20. A system, comprising:
a virtual imaging device;
an energy delivery device in communication with the virtual imaging device, the energy delivery device configured to deliver energy to an area of interest within a gastrointestinal lumen of a patient identified by the virtual imaging device to disrupt at least a portion of an undesired tissue.

21. The system of claim 20, wherein the energy delivery device is configured to deliver acoustic energy to the area of interest.

22. The system of claim 20, wherein the energy delivery device is configured to deliver electromagnetic energy to the area of interest.

23. The system of claim 20, wherein the virtual imaging device is a computed tomography scanner.

24. The system of claim 20, wherein the virtual imaging device is a magnetic resonance imaging device.
